(19) United States
(12) Patent Application Publication
(10) Pub. No.: US 2023/0346849 A1
(43) Pub. Date: Nov. 2, 2023

(54) Appetite Suppressant and Method of Use

(71) Applicant: Teresa Bono, Toronto (CA)

(72) Inventor: Teresa Bono, Toronto (CA)

(21) Appl. No.: 17/732,831

(22) Filed: Apr. 29, 2022

(51) Int. Cl. A61K 35/57; A61K 35/20; A61K 47/46; A61K 31/7004; A61K 9/00; A61K 36/87; A23L 33/00; A23L 33/125; A23L 33/19; A23F 5/24; C12G 1/00

(52) U.S. Cl. CPC A61K 35/57; A61K 35/20; A61K 47/46; A61K 31/7004; A61K 9/0095; A61K 36/87; A23L 33/40; A23L 33/125; A23L 33/19; A23F 5/243; C12G 1/00; A23V 2002/00; C12G 2200/21

(57) ABSTRACT

The present invention provides an appetite suppression composition added to coffee, whereby the composition includes a synergistic combination of egg yolk protein sugar and milk protein that imparts appetite suppression over 4-6 hours.

### BACKGROUND OF THE INVENTION

People are constantly seeking safe and effective appetite suppressants. Pharmaceutical options exist but often come with side effects. Numerous nutritional supplements are commercially available but they are unregulated and there is a sad history of permeant damage with prolonged use. One well-known suppressant is the simple cup of coffee. While generally effective, it has a drawback of digestive agitation and irregular effectiveness. There is a need for a safe and effective simple appetite suppressant.

The present invention addresses this issue.

### SUMMARY OF THE INVENTION

While coffee is now available in many flavors and with many combinations of ingredients, none of the known combinations both suppress the appetite and provide for a drink imparting minimal digestive agitation.

The present invention has discovered a suppressant composition having a unique ratio imparting a synergistic effect whereby safe and effective appetite suppression is achieved.

The invention relies on the critical synergistic ratio of the suppressant composition whereby the ratio imparts the desired results of appetite suppression of 4-6 hours and further reduces known digestive agitation of coffee alone.

The additives are a suppressant composition of egg yolk, sugar, and milk protein.

The ratio discovered in the present application is presented:
Egg yolk protein:sugar:milk protein

In one embodiment, the ratio is
1:3.5-5.5:2.5-3.0

In another embodiment, the ratio is
1:3.8-5.2:2.8-3.0 The ratio is shown to be unique and provide the desired results of appetite suppression and ease of digestion.

In one embodiment, the invention is a composition for suppressing appetite comprising:
a suppressant composition consisting of egg yolk, sugar and milk protein, whereby the ratio of egg yolk protein:sugar:milk protein is 1:3.5-5.5:2.5-3.0;
said suppressant composition added to coffee q.s. of 160 ml. In one embodiment, the ratio of egg yolk protein:sugar:milk protein is 1:3.8-5.2:2.8-3.0.

In one embodiment, the coffee is provided as 51 ml espresso and q.s. water to 160 ml total.

In one embodiment, the coffee is provided as 51 ml espresso and q.s. water to 160 ml total.

In one embodiment, the invention is a composition for suppressing appetite consisting of:
a suppressant composition consisting of egg yolk, sugar and milk protein, whereby the ratio of egg yolk protein:sugar:milk protein is 1:3.5-5.5:2.5-3.0;
said suppressant composition added to coffee q.s. of 160 ml.

In one embodiment, the invention is a composition for suppressing appetite consisting of:
a suppressant composition consisting of egg yolk, sugar and milk protein, whereby the ratio of egg yolk protein:sugar:milk protein is 1:3.8-5.2:2.8-3.0; and
said suppressant composition added to 14-21 ml of red wine with an alcohol content between 12% and 15% and coffee q.s. of 160 ml.

In one embodiment, the invention is a method of providing appetite suppression for 4-6 hours, said method comprising the steps of:
preparing a composition as disclosed herein;
administering said composition to a person, whereby said administering includes complete consumption of 160 ml in 10-15 minutes.

In one embodiment, the invention is a method of providing appetite suppression for 4-6 hours, said method consisting of the steps of:
preparing a composition as disclosed herein;
administering said composition to a person, whereby said administering includes complete consumption of 160 ml in 10-15 minutes.

### DETAILED DESCRIPTION OF THE PREFERRED EMBODIMENTS

The present invention is an appetite suppressant added to coffee. While coffee alone is known to have some appetite suppressant effects, it often imparts a caffeine crash and digestive agitation. The present invention addresses these shortcomings.

The suppressant composition is a single egg yolk that has approximately 2.7 g protein and 4.51 g fat.

A single yolk is combined with between 10.35 g and 14.3 g of sugar and 49 ml milk, whereby the milk has 8-8.5 g protein. This mixture is combined with coffee to a quantity of 160 ml total.

In one embodiment, the coffee is 51 ml espresso with q.s. water to 160 ml total.

In this system, a synergistic ratio of Egg yolk protein:sugar:milk protein is present and imparts appetite suppression 4-6 hours and reduces digestive effects normally associated with coffee drinks.

In one embodiment, it has been discovered the addition of red wine introduces tannins that enhance the synergistic effect.

The MCP (methyl cellulose precipitable) tannin assay is commonly used to determine tannin level in red wine. Red wine with an alcohol content between 12% and 15% typically exhibit MCP measured Tannin levels between 0.5-4.0 g/l.

The present invention has discovered addition of between 14.00 ml to 22.00 ml of red wine with an alcohol content between 12% and 15% enhances the synergistic effect of the suppressant composition.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A composition for suppressing appetite comprising:
a suppressant composition consisting of egg yolk, sugar and milk protein, whereby the ratio of egg yolk protein:sugar:milk protein is 1:3.5-5.5:2.5-3.0;
said suppressant composition added to coffee q.s. of 160 ml.

2. The composition of claim 1 whereby the ratio of egg yolk protein:sugar:milk protein is 1:3.8-5.2:2.8-3.0.

3. The composition of claim 1 whereby coffee is provided as 51 ml espresso and q.s. water to 160 ml total.

4. The composition of claim 2 whereby coffee is provided as 51 ml espresso and q.s. water to 160 ml total.

5. A composition for suppressing appetite consisting of:
a suppressant composition consisting of egg yolk, sugar and milk protein, whereby the ratio of egg yolk protein:sugar:milk protein is 1:3.8-5.2:2.8-3.0; and
said suppressant composition added to 14-21 ml of red wine with an alcohol content between 12% and 15% and coffee q.s. of 160 ml.

6. A method of providing appetite suppression for 4-6 hours, said method comprising the steps of:
preparing a composition of claim 1;
administering said composition to a person, whereby said
administering includes complete consumption of 160 ml in 10-15 minutes.
